(19) United States
(12) Patent Application Publication
(10) Pub. No.: US 2007/0281046 A1
(43) Pub. Date: Dec. 6, 2007

(54) Method for removing organic contaminants from Chinese herb medicines

(75) Inventors: Hsiao-Wei Wen, Taichung (TW); Fong-In Chou, Hsinchu (TW)

Correspondence Address: BACON & THOMAS, PLLC, 625 SLATERS LANE, FOURTH FLOOR, ALEXANDRIA, VA, 22314, US

(73) Assignee: Committee on Chinese Medicine and Pharmacy, Department of Health, Executive Yuan, Taipei City (TW)

(21) Appl. No.: 11/790,116

(22) Filed: Apr. 24, 2007

(30) Foreign Application Priority Data
May 30, 2006 (TW) 95119221

(51) Int. Cl. A61K 36/00

(52) U.S. Cl. 424/774

(57) ABSTRACT

The present invention relates to the degradation and removal of organochlorine pesticides (OCP) from Chinese herb medicines. In this invention, gamma irradiation is used for the degradation and removal of OCP, such as Pentachloronitrobenzene, Aldrin, Lindane, Heptachlor, Endosulfan-1, Endosulfan-2, o,p′-DDD, p,p′-DDD, p,p′-DDE, and p,p′-DDT from Chinese herb medicines. The method of the present invention requires no chemical reagents, and the application of the method is not limited by the volume and morphology of the Chinese herb medicines.

### FIELD OF THE INVENTION

The invention relates to the removal of residual organic contaminants, such as organochlorine pesticides (OCP), from Chinese herb medicines by the application of gamma irradiation and method thereof.

### BACKGROUND OF THE INVENTION

Currently, the concentration of agricultural chemicals remaining in many of the Chinese herb medicines sold on the market is found to be over the acceptable value. For example, for the Chinese herb medicine Panax quinquefolium, the organochlorine pesticide pentachloronitrobenzene (PCNB) is more likely to remain in the herb than other pesticides. Though the pesticides persistently remain in the Chinese herb medicines could seriously affect the health of the users who take the medicines, methods that can remove a large proportion of the pesticides remaining in the Chinese herb medicines in an effective and easy fashion have not been reported so far. A major obstacle exists in that a majority of the Chinese herb medicines are desiccated plant tissues, and pesticides tend to remain on the surface or in the internal tissue of the herbs, which makes the process of removing the pesticides difficult. For instance, P. quinquefolium is a perennial plant, and if organochlorine pesticides were sprayed thereon, the pesticides tend to accumulate inside of the plant tissues, and this renders the method of washing ineffective for removing the organochlorine pesticides. Although the method of supercritical fluid extraction has been utilized to remove the pesticides remaining in the Chinese herb medicines of powder form, the method is still ineffective for removing the organochlorine pesticides from the Chinese herb medicines that are morphologically and volumetrically larger.

Gamma irradiation has been used to degrade contaminants in soils previously, but gamma irradiation has not been applied for degrading and removing the accumulated organic contaminants from the Chinese herb medicines so far. The use of gamma irradiation for eliminating microbes in the Chinese herb medicines has been described in many research literatures, such as the one published by one of the co-inventors of the invention; Dr. Chou Fong-In, and colleagues in Nuclear Science Journal, vol. 38, No. 4, pp. 279-288 (August, 2001). In this report, gamma irradiation was used to sterilize thirty different types of Chinese herb medicines, and the result indicated that gamma irradiation could completely eliminate the microbes in the herbs tested, and there was no significant effects on the composition of the medicines, neither did the irradiated samples of the tested herbs show any signs of radioactivity, which makes this method ideal for preserving the Chinese herb medicines. The thirty different types of Chinese herb medicines included Scutellariae Radix, Ginkgo Semen, Rehmanniae Radix, prepared Rehmanniae Radix, Lycii Fructus, Ginseng Radix, Cnidii Rhizoma, Codonopsitis Pilosulae Radix, Dioscoreae Rhizoma, Anagelicae Sinensis Radix, Salivae miltiorrhiaze Radix, Platycodi Radix, Armeniacae Semen, Crataegi Fructus, Pericarpium Citri Reticulatae, Ophiopogonis Tuber, Poriae Sclerotium, Lonicerae Flos, Angelicae Dahurica Radix, Atractylodis Rhizoma, Fritillaria Thunbergii Bulbus, Fritillariae Cirrhosae Bulbus, Schisandrae Fructus, Lilii Bulbus, Rhei Rhizoma, Polygonum Cuspidatum, Lithospermi Radix, Bupleuri Radix, Corni Fructus and Astragali Radix.

### SUMMARY OF THE INVENTION

A major objective of the invention is to propose a method for removing organic contaminants from contaminated Chinese herb medicines, especially a method that utilizes gamma irradiation for removing organic contaminants from the contaminated Chinese herb medicines.

According to the invention, the method for removing organic contaminants from the Chinese herb medicines that have been contaminated thereof comprising:

a) Confirming whether Chinese herb medicines contain organic contaminants;

b) Irradiating the contaminated Chinese herb medicines with gamma radiation, so as to remove a proportion of the organic contaminants from the contaminated Chinese herb medicine.

Preferably, the Chinese herb medicines receive gamma irradiation in dosages ranging from 5 to 25 kGy in step a). More preferably, the gamma irradiation has a dose rate ranging from 0.2 to 3.0 kGy/hr.

Preferably, the organic contaminants are agricultural chemicals. The agricultural chemicals may be pesticides or herbicides. The agricultural chemicals include (but not limited thereto) organochlorine chemicals, for examples pentachloronitrobenzene (PCNB), Aldrin, Lindane, Heptachlor, Endosulfan-1, Endosulfan-2, o,p′-DDD, p,p′-DDD, p,p′-DDE, and p,p′-DDT.

Preferably, the gamma irradiation removes the organic contaminants contained in the Chinese herb medicines therefrom by more than 50% in quantity.

Preferably, step a) comprises the use of gas chromatography analysis, or gas chromatography together with mass spectrometry analysis.

### BRIEF DESCRIPTION OF DRAWINGS

The structure and the technical means adopted by the present invention to achieve the above and other objectives can be best understood by referring to the following detailed description of the preferred embodiments and the accompanying diagrams, wherein:

FIG. 1 shows the effects of gamma irradiation on the degradation of organochlorine pesticide, pentachloronitrobenzene (PCNB), in which the hollow circle (∘) indicates a subject was irradiated in 90% aqueous methanol solution, while the filled circle () indicates a subject was irradiated in a sample of P. quinquefolium.

FIG. 2 shows the L929 cell toxicity of P. quinquefolium samples that contain PCNB before and after gamma irradiation.

### DETAILED DESCRIPTION OF PREFERRED EMBODIMENTS

The invention proposes a method of using gamma irradiation for the degradation and removal of residual organic contaminants (such as agricultural chemicals) in Chinese herb medicines. The method of the invention not only degrades and removes residual organic contaminants in Chinese herb medicines, it does not require any addition of chemical reagents, and its application is not limited by the volume and morphology of the Chinese herb medicines.

In a preferred embodiment of the invention, samples of P. quinquefolium that contained 0.5 ppm of nine organochlorine pesticides were subjected to gamma irradiation; the nine organochlorine pesticides were Aldrin, Lindane, Heptachlor, Endosulfan-1, Endosulfan-2, o,p′-DDD, p,p′-DDD, p,p′-DDE, and p,p′-DDT. Subsequently, it was found that the concentration of each of the organochlorine pesticides decreased as the dosage of gamma irradiation increased, and after receiving a dosage of 20 kGy, the remaining concentration of each of the organochlorine pesticides dropped below 0.15 ppm.

In another preferred embodiment of the invention, samples of P. quinquefolium with the organochlorine pesticide PCNB, which is more likely to persistently remain in Chinese herb medicines than other pesticides, were subjected to gamma irradiation, and it was discovered that the concentration and toxicity of PCNB decreased as the dosage of gamma irradiation increased. When samples of 2 ppm PCNB in methanol solution underwent gamma irradiation at dosages of 20 kGy and 25 kGy, the concentration of the remaining PCNB were 2.79% (0.056 ppm) and 0.86% (0.008 ppm) as compared to the concentration of PCNB in methanol solution before receiving treatment.

After subjecting samples of P. quinquefolium containing 2 ppm of PCNB to gamma irradiation treatment, the results showed that the PCNB concentration in P. quinquefolium samples that received 20 kGy of gamma irradiation was reduced to 4.01% (0.08 ppm) of the original concentration, which indicated that gamma irradiation is effective for degrading the organochlorine pesticide PCNB. Moreover, the result from the assays using L929 mouse fibroblast cells revealed that after being treated by gamma irradiation, the extracted solution of P. quinquefolium samples that contained PCNB has lower cell toxicity than those from samples that have not been irradiated. Gamma irradiation of 10 kGy and 15 kGy in dosages would not affect the concentration of marker components like ginsenosides, which includes Rb1, Rg1, Rc, Rd, and Re in P. quinquefolium. After receiving 20 kGy of gamma irradiation, the concentration of the aforesaid key ingredients in the samples was 90-94% as compared to the concentration before receiving irradiation.

### EXAMPLES

1. The Degradation of Nine Types of Organochlorine Pesticides by Gamma Irradiation in Samples of P. quinquefolium.

The nine organochlorine pesticides that included Aldrin, Lindane, Heptachlor, Endosulfan-1, Endosulfan-2, o,p′-DDD), p,p′-DDD, p,p′-DDE, and p,p′-DDT were simultaneously prepared in a n-hexane solution. Subsequently, the n-hexane solution that contained the aforesaid organochlorine pesticides was evenly sprayed into samples of P. quinquefolium; the concentration of each of the nine organochlorine pesticides contained in the samples was approximately 0.5 ppm. The samples were cut into pieces with scissors, and then divided into separate packs that weighed 10 g, respectively; the packs were subjected to gamma irradiation of different dosages ranging from 0, 5, 10, 15, 20, and 25 kGy, and followed by the analysis of concentration of the aforesaid organochlorine pesticides. Each dosage of gamma irradiation was repeated three times, and the packs that did not undergo gamma irradiation were used as the control group. Solution was extracted from the samples by using 70% aqueous acetone, and then the extracted solution was subjected to filtration, a decrease in pressure, and condensation in order to remove acetone, followed by the addition of hydrochloric acid and n-hexane to bring the extracted solution to 100 ml, so that the organochlorine pesticides were distributed to the organic layer. Consequently, magnesium silicate was added into the resulted solution in order to depigment and purify it, and once the solution has undergone a decrease in pressure and condensation again for drying, it was brought to 5 ml by adding n-hexane solution to prepare it for gas chromatography (GC) analysis. After undergoing centrifugation, 2 μl of the resulted solution was taken for GC analysis to find out the respective concentration of each of the aforesaid organochlorine pesticides remaining in the samples after receiving various dosages of gamma irradiation.

As shown in Table 1, the concentration of all of the aforesaid organochlorine pesticides showed significant decrease relative to the increase in the dosages of gamma irradiation. The treatment of 20 kGy of gamma irradiation reduced the remaining concentration of all of the organochlorine pesticides to below 0.2 ppm, whereas treatment of 25 kGy of gamma irradiation reduced the remaining concentration of all of the organochlorine pesticides to below 0.15 ppm. The rate of degradation for Lindane and p,p′-DDT was higher than that for the other organochlorine pesticides; in which 84% of Lindane and p,p′-DDT were degraded.

TABLE 1
The remaining concentration (ppm)^{a)} of organochlorine pesticides in
samples after receiving gamma irradiation.

| Organochlorine | Dosages of Gamma Irradiation (kGy) | | | | | |
|---|---|---|---|---|---|---|
| Pesticides | 0 | 5 | 10 | 15 | 20 | 25 |
| Aldrin | 0.532^{a)} | 0.376 | 0.279 | 0.218 | 0.168 | 0.123 |
| Lindane | 0.549 | 0.383 | 0.278 | 0.206 | 0.147 | 0.089 |
| Heptachlor | 0.532 | 0.421 | 0.319 | 0.254 | 0.195 | 0.142 |
| Endosulfan-1 | 0.519 | 0.392 | 0.311 | 0.251 | 0.192 | 0.140 |
| Endosulfan-2 | 0.515 | 0.359 | 0.263 | 0.198 | 0.144 | 0.093 |
| o,p′-DDD | 0.476 | 0.337 | 0.250 | 0.196 | 0.155 | 0.119 |
| p,p′-DDD | 0.502 | 0.370 | 0.282 | 0.227 | 0.179 | 0.147 |
| p,p′-DDE | 0.544 | 0.404 | 0.313 | 0.249 | 0.195 | 0.145 |
| p,p′-DDT | 0.520 | 0.344 | 0.242 | 0.178 | 0.127 | 0.082 |

^{a)}The values were the mean derived from values obtained after carrying out each dosage of gamma irradiation; each dosage of gamma irradiation was repeated three times.

2. The Concentration of PCNB and Ginsenoside Remaining in Samples of P. quinquefolium after Receiving Gamma Irradiation.

a. The PCNB Concentration in Samples of P. quinquefolium

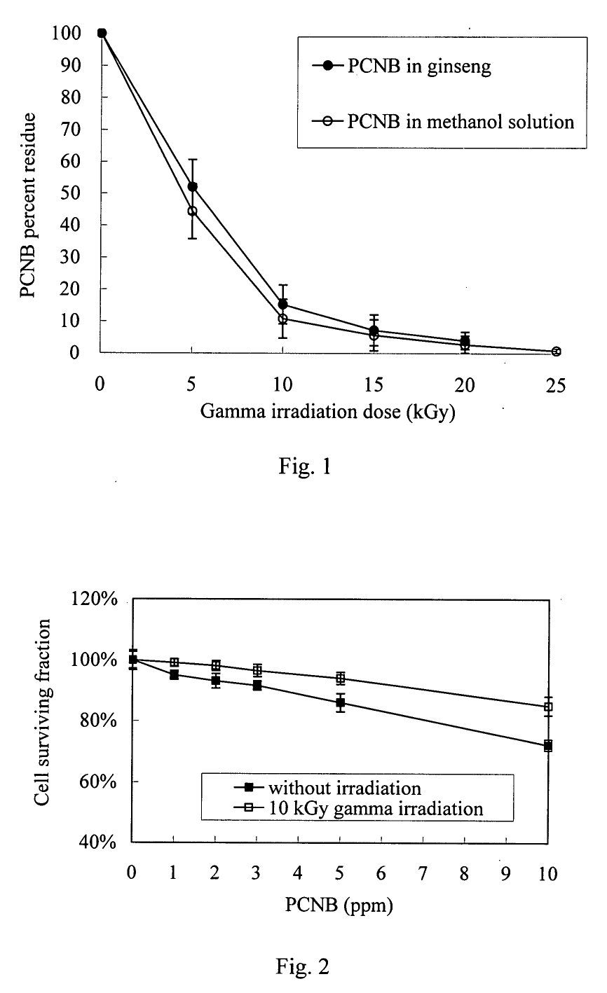

The PCNB to be tested was prepared in 100% methanol solution, and the methanol solution that contained PCNB was evenly sprayed into samples of P. quinquefolium; the concentration of PCNB contained in the samples was approximately 2 ppm. The samples were cut into pieces with scissors, and then divided into separate packs that weighed 10 g, respectively; the packs were subjected to gamma irradiation of different dosages ranging from 0, 5, 10, 15, 20, and 25 kGy, and followed by the analysis of concentration of PCNB. Because the percentage of water in P. quinquefolium was approximately 10%, PCNB was also prepared in 90% aqueous methanol solution and subjected to gamma irradiation for serving as the control group. The results are shown in FIG. 1, which indicated that gamma irradiation could effectively degrade and remove PCNB from samples of P. quinquefolium.

b. The Concentration of Marker Components in Samples of P. quinquefolium

Samples of P. quinquefolium that have respectively received gamma irradiation in dosages of 0, 10, 15, and 20 kGy were analyzed to find out the concentration of marker components therein, with the samples that have not received gamma irradiation as the control group. To extract marker components from the samples for analysis, 10 g of the samples were taken and prepared in 50 ml of 70% aqueous methanol solution, and heated in a convection heater at 60° C. for 15 minutes, followed by centrifugation in order to obtain the extract solution. Subsequently, the residual of the samples were subjected to the above-mentioned procedure again, and the extracted solution from the two rounds of procedures were mixed together and filtered, followed by the addition of 70% aqueous methanol solution to bring the volume up to 100 ml; the resulted solution was then stored at −20° C. and analyzed within 3 days. Before carrying out the analysis of ingredients, an adequate amount of the resulted solution was underwent filtration through a 0.22 μm pore size membrane, and then 20 μl of the solution was injected into a high performance liquid chromatographer for analysis (HPLC, Waters model 2695) each time; the analyses were carried out in combination with UV light—Visible light sensor of Waters model 2487, and a silicon-based reverse phase C-18 column (Cosmosil 5C18-MS-II, 5 μm, 4.6×250 mm, Nacalai Tesque, Inc., Kyoto, Japan) was used for analyzing the marker components of P. quinquefolium in the solution. The marker components of P. quinquefolium tested in the experiment included ginsenoside Rb1, ginsenoside Rg1, ginsenoside Rc, ginsenoside Rd, and ginsenoside Re; the detection wavelength of UV light was 203 ηm. The solution of 15% aqueous phosphoric acid (H₃PO₄)—acetonitrile (CH₃CN) (81:19 to 60:40) was used as the moving phase, and the moving phase was analyzed at 25° C. and 1.0 ml/min of flow velocity after being filtered with a 0.45 μm filtration membrane, so as to measure the concentration of the marker components in samples of P. quinquefolium that received various dosages of gamma irradiation.

Table 2 shows the concentration of ginsenoside Rb1, ginsenoside Rg1, ginsenoside Rc, ginsenoside Rd, and ginsenoside Re obtained by HPLC from P. quinquefolium samples without gamma irradiation, and the ones that received dosages in 10, 15, 20 kGy. It was discovered that gamma irradiation at dosages of 10 and 15 kGy did not significantly affect the concentration of ginsenosides in P. quinquefolium samples, while gamma irradiation at dosage of 20 kGy reduced the concentration of the five marker components in P. quinquefolium samples by 6 to 10%. As a result, the outcome indicated that gamma irradiation does not significantly affect the concentration of ginsenosides in P. quinquefolium, which supports the use of gamma irradiation as an effective method for reducing the concentration of the aforesaid organochlorine pesticides in P. quinquefolium.

TABLE 2
The analysis of the concentration of marker components of
P. quinquefolium before and after receiving gamma irradiation.

| Ginsenoside | Dosages of Gamma Irradiation (kGy) | | | |
|---|---|---|---|---|
| (mg/g) | 0 | 10 | 15 | 20 |
| Rb1 | 31.37 ± 0.15^{a)} | 31.01 ± 0.11 | 30.59 ± 0.63 | 29.39 ± 0.36 |
| Rc | 16.12 ± 0.47 | 15.91 ± 0.43 | 15.08 ± 0.27 | 14.46 ± 0.28 |
| Rd | 9.02 ± 0.32 | 8.77 ± 0.15 | 8.44 ± 0.25 | 8.20 ± 0.17 |
| Re | 15.98 ± 0.50 | 15.79 ± 0.21 | 15.04 ± 0.33 | 14.53 ± 0.14 |
| Rg1 | 1.56 ± 0.09 | 1.56 ± 0.06 | 1.47 ± 0.03 | 1.43 ± 0.02 |

^{a)}The values were the mean and the standard deviation derived from values obtained after carrying out each dosage of gamma irradiation; each dosage of gamma irradiation was repeated three times.

3. The Suppression of Cell Toxicity of the Organochlorine Pesticide PCNB by Gamma Irradiation

The cell toxicity of the samples of P. quinquefolium containing PCNB before and after receiving gamma irradiation was tested by the MTT (3-(4,5-dimethylthiazol-2-yl)-2,5-diphenyltetrazolium bromide) method. Accordingly, cells were taken from L929 cell line and quantitatively cultivated in 24-well culture dishes. After replacing fresh culture medium (800 μl/well) for the L929 cells cultivated in the 24-well culture dishes, the extracted solution derived from P. quinquefolium samples containing PCNB that received either no gamma irradiation or 10 kGy of gamma irradiation was diluted and added into the culture dishes; 200 μl of the extracted solution was added so as to make the final concentration of P. quinquefolium in the culture media to be 5 mg/ml. The concentration of PCNB in the culture media was 0, 1, 2, 3, 5 and 10 ppm, respectively; and the culture cells were incubated for 2 days at 37° C. and 5% CO₂. During the incubation, the culture medium was not replaced, and the test regarding each concentration was carried out by using 4 wells at a time, with wells that have been added extracted solution of P. quinquefolium without PCNB as the control group. After incubating for 2 days, the cells were subjected to cell toxicity test by using the MTT method. Firstly, the cells were washed with PBS twice, and then added with 900 μl of culture medium and 100 μl of freshly prepared MTT solution (5 mg/ml). Meanwhile, wells without cells but added with the identical culture medium and MTT solution described above were used to obtain the background value; after incubating for 4 hours, the upper clear solution was separated and discarded, and then added 1 ml of dimethyl sulfoxide into each well. Subsequently, the absorption value of the wells with a wavelength at 570 ηm was obtained by using an ELISA reader. FIG. 2 shows the outcome of cell toxicity test from cells treated with the extracted solution of P. quinquefolium that contained various concentration of PCNB after 2 days of incubation, as found out by the MTT method. The absorption value of the wells added with extracted solution of P. quinquefolium without PCNB was designated as 100%, and then the cell survival rate for wells that received extracted solutions of P. quinquefolium of different PCNB concentrations was calculated. It was revealed that for the P. quinquefolium samples containing PCNB, the cell toxicity of the solution extracted therefrom that received gamma irradiation was lower than that of the one without gamma irradiation. For the P. quinquefolium samples that contained a concentration of PCNB as high as 10 ppm, the solution extracted therefrom is toxic to cells. However, after receiving 10 kGy of gamma irradiation, the cell toxicity of the solution extracted from the aforesaid P. quinquefolium samples containing PCNB could be effectively reduced.

The present invention has been described with preferred embodiments thereof, and it is understood that many changes and modifications to the described embodiments can be carried out without departing from the scope and the spirit of the invention that is intended to be limited only by the appended claims.

1. A method for removing organic contaminants from contaminated Chinese herb medicines, comprising:
irradiating the Chinese herb medicines contaminated with organic contaminants with gamma radiation, so as to remove a portion of the organic contaminants from the contaminated Chinese herb medicines.

2. The method of claim 1, wherein the Chinese herb medicines receive gamma irradiation in dosages ranging from 5 to 25 kGy.

3. The method of claim 2, wherein the gamma irradiation has a dose rate ranging from 0.2 to 3.0 kGy/hr.

4. The method of claim 1, wherein the organic contaminants are agricultural chemicals.

5. The method of claim 4, wherein the agricultural chemicals are organochlorine chemicals.

6. The method of claim 5, wherein the organochlorine chemicals are pentachloronitrobenzene (PCNB).

7. The method of claim 4, wherein the gamma irradiation removes the organic contaminants contained in the Chinese herb medicines therefrom by more than 50% in quantity.

8. The method of claim 4 further comprising a step of confirming the presence of organic contaminants in the Chinese herb medicines prior to carrying out the gamma irradiation.

9. The method of claim 8, wherein the step of confirming the presence of organic contaminants in the Chinese herb medicines comprises the use of gas chromatography analysis, or gas chromatography together with mass spectrometry analysis.
